(19) United States
(12) Patent Application Publication
(10) Pub. No.: US 2016/0302957 A1
(43) Pub. Date: Oct. 20, 2016

(54) ORTHOPEDIC WALKER BOOT HAVING AN OUTER SOLE FORMED FROM MULTIPLE MATERIALS

(71) Applicant: Ortho Systems d/b/a Ovation Medical, Agoura Hills, CA (US)

(72) Inventors: Joseph Michael IGLESIAS, Agoura Hills, CA (US); Tracy E. GRIM, Thousand Oaks, CA (US)

(21) Appl. No.: 15/167,956

(22) Filed: May 27, 2016

Related U.S. Application Data
(63) Continuation-in-part of application No. 13/441,552, filed on Apr. 6, 2012, now abandoned.
(60) Provisional application No. 61/472,946, filed on Apr. 7, 2011.

(51) Int. Cl. A61F 5/058; A61F 5/01; A43B 7/32; A43B 13/12; A43B 3/02; A43B 7/14

(52) U.S. Cl. CPC A61F 5/0585; A43B 3/02; A43B 7/141; A43B 7/32; A43B 13/122; A61F 5/0127

(57) ABSTRACT

Aspects of an orthopedic walking boot are disclosed. The walking boot includes a base supporting a user's foot. The orthopedic walking boot includes a support assembly extending from the base to support the user's lower leg. The orthopedic walking boot includes an outer sole including a walking surface having first and second materials. The second material has a greater shock absorbing characteristic less than the first material along at least a portion of the outer sole.

### CROSS-REFERENCE TO RELATED APPLICATION(S)

This application claims the benefit of U.S. Non-Provisional Patent Application Ser. No. 13/441,552 entitled “REMOVABLE LEG WALKING BOOT,” filed on Apr. 6, 2012, which claims the benefit of U.S. Provisional Patent Application No. 61/472,946, entitled “Removable Leg Walking boot,” filed on Apr. 7, 2011. The contents of U.S. patent application Ser. Nos. 13/441,552 and 61/472,946 are expressly incorporated by reference herein in their entirety.

### BACKGROUND

Walking boots have been increasingly used as a replacement to casts in treating injuries that require immobilization for an extended period of time to heal. A walking boot provides complete immobilization to the toes, forefoot, mid foot, ankle and lower leg in multiple planes. This means the foot and lower leg are fixed to 90 degree position for, oftentimes 4 to 6 weeks.

In the past, plaster or fiberglass casts were used to treat these types of injuries however walking in a cast is cumbersome and sometimes painful because of the heel formed in the plaster cast. Oftentimes the plaster cast heel would add up to 3 inches of height to the immobilized limb resulting in a leg length discrepancy. For instance, the additional 3 inches of leg length would create an asymmetry. Attempting to walk with an interrupted, elevated surface is difficult and at times unsafe. As a result, the patient might end up dragging the injured leg behind him/her, walk on crutches, or end up confined to a wheelchair just to be comfortably mobile.

Walking boots were designed to provide a more comfortable form of immobilization. Walking boots provide a lower center of gravity, which allows for a gait that is more natural than when walking in a cast. As a result, an injured patient could be somewhat mobile while having the foot, ankle and lower leg remain immobilized, and set at a 90 degree angel without any ability to pronate or supinate.

Walking boots are typically designed to be as low to the ground as possible to provide a more comfortable gait. In fact walking boots are typically designed to match the height of the opposing foot to avoid a leg length discrepancy. A leg length discrepancy is an asymmetrical pelvic, tibia or femur length. One cause of leg length discrepancy is uneven or unleveled footwear height. As a result, the gait pattern will present as a pelvic dip to the shortened side when standing. The opposite leg is likely to increase its knee and hip flexion to reduce its length. This results in an altered gait.

Since walking boots freeze the foot and ankle in all planes, while also inducing heel height asymmetry, the normal gait accommodations described above are altered and patients are thrown into an emergent accommodative gait phase to keep safe balance when attempting to walk.

Normal gait cycle has multiple phases known as heel strike, mid stance, and push off. The walking boot bottom has to serve as a replacement for the injured foot by providing a stable platform for the body to swing forward while balanced on the walking boot bottom without interruption. The rigid plastic frame is often formed with a rocker bottom extending from the front to the back of the walker, thus allowing the patient to heel strike and rock forward while balancing the body from the knee and the hip. The outer sole of the walker is made of a thin softer material that will provide traction and some form of anti-slip while walking on smooth or wet surfaces. The softer material must nonetheless be fairly rigid to maintain a continuous slope for a non-interrupted gait. As a result, orthopedic walkers do not absorb shock well.

Heretofore, it was not practical to design an outer sole for an orthopedic boot that absorbs shock. A thicker material for shock absorption could compress, and thereby, interrupt the normal gait. Accordingly, what is needed, is an outer sole having additional material that will allow for cushioning in specific areas to absorb shock over the rigid frame. The materials comprising the outer sole must be engineered to have limited compression so the patient will have a continuous flowing gait with no stutter or step-off when the weight of the patient is applied to that boot.

### SUMMARY

Aspects of an orthopedic walking boot are disclosed. The walking boot includes a base supporting a user's foot. The orthopedic walking boot includes a support assembly extending from the base to support the user's lower leg. The orthopedic walking boot includes an outer sole including a walking surface having first and second materials. The second material has a greater shock absorbing characteristic less than the first material along at least a portion of the outer sole.

It is understood that other aspects of orthopedic walking boots will become readily apparent to those skilled in the art from the following detailed description, wherein various aspects are shown and described by way of illustration. As will be realized, these aspects may be implemented in other and different forms and its several details are capable of modification in various other respects. Accordingly, the drawings and detailed description are to be regarded as illustrative in nature and not as restrictive

### BRIEF DESCRIPTION OF THE DRAWINGS

Various aspects of orthopedic walking boots will now be presented in the detailed description by way of example, and not by way of limitation, with reference to the accompanying drawings, wherein:

FIG. 1 is a cross-section view of an exemplary embodiment of an orthopedic walking boot in contact with a surface.

FIG. 2 is a cross-section view of the boot of FIG. 1.

FIG. 3A is a cross-section view of another exemplary embodiment of an orthopedic walking boot.

FIG. 3B is a side view of a portion of the orthopedic walking boot of FIG. 3A.

FIG. 4 is a cross-section of the boot of FIG. 3A in partial contact with a surface.

FIG. 5 is a side view of an exemplary embodiment of an orthopedic walking boot comprising an outer sole comprising a plurality of materials.

FIG. 6 is a side view of an exemplary embodiment of an orthopedic walking boot comprising an outer sole comprising a plurality of materials.

FIG. 7 is a side view of an exemplary embodiment of an orthopedic walking boot comprising the same plurality of materials described with respect to FIG. 6.

FIG. 8 is a side view of an exemplary embodiment of an orthopedic walking boot comprising the same plurality of materials described with respect to FIG. 6.

FIG. 9 illustrates an exemplary embodiment of regulating the ratio of harder material pellets in an injection machine to form the outer sole of the orthopedic walking boot.

FIG. 10 illustrates an exemplary embodiment of injecting the first and second materials through different injector nozzles.

FIG. 11 is a side view of an exemplary embodiment of an orthopedic walking boot having an outer sole comprising a plurality of materials.

FIG. 12 is a side view of an exemplary embodiment of an orthopedic walking boot having an outer sole comprising a plurality of materials.

FIG. 13 is a side view of an exemplary embodiment of an orthopedic walking boot having a plurality of materials in an outer sole.

FIG. 14 is a side view of an exemplary embodiment of an orthopedic walking boot having an outer sole comprised of a plurality of materials.

FIG. 15 is a side view of an exemplary embodiment of an orthopedic walking boot having an outer sole comprised of a plurality of materials.

FIG. 16 is a side view of an exemplary embodiment of an orthopedic walking boot having an outer sole comprised of a plurality of materials.

FIG. 17 is a side view of an exemplary embodiment of an orthopedic walking boot having an outer sole comprised of a plurality of materials.

FIGS. 18a-c are side views of an exemplary embodiment of an orthopedic walking boot having an outer sole comprised of a composition of rubber materials.

FIG. 19 is a side view of an exemplary embodiment of an orthopedic walking boot having an outer sole comprised of a plurality of materials.

FIG. 20 is a side view of an exemplary embodiment of an orthopedic walking boot having an outer sole comprised of a plurality of materials.

### DETAILED DESCRIPTION

The detailed description set forth below in connection with the appended drawings is intended as a description of various exemplary embodiments of the present invention and is not intended to represent the only embodiments in which the present invention may be practiced. The detailed description includes specific details for the purpose of providing a thorough understanding of the present invention. However, it will be apparent to those skilled in the art that the present invention may be practiced without these specific details. In some instances, well-known structures and components are shown in block diagram form in order to avoid obscuring the concepts of the present invention. Acronyms and other descriptive terminology may be used merely for convenience and clarity and are not intended to limit the scope of the invention

The word “exemplary” is used herein to mean serving as an example, instance, or illustration. Any embodiment described herein as “exemplary” is not necessarily to be construed as preferred or advantageous over other embodiments. Likewise, the term “embodiment” of an apparatus does not require that all embodiments of the invention include the described components, structure, features, functionality, processes, advantages, benefits, or modes of operation.

The terms “connected,” “coupled,” or any variant thereof, mean any connection or coupling, either direct or indirect, between two or more elements, and can encompass the presence of one or more intermediate elements between two elements that are “connected” or “coupled” together.

Any reference to an element herein using a designation such as “first,” “second,” and so forth does not generally limit the quantity or order of those elements. Rather, these designations are used herein as a convenient method of distinguishing between two or more elements or instances of an element. Thus, a reference to first and second elements does not mean that only two elements can be employed, or that the first element must precede the second element.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and this disclosure.

The terms “durometer” and “hardness” may be used interchangeably throughout the disclosure, but carry the same meaning. Durometer is the measurement of the hardness of a material. A material's hardness may be defined as the material's resistance to permanent indentation.

It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

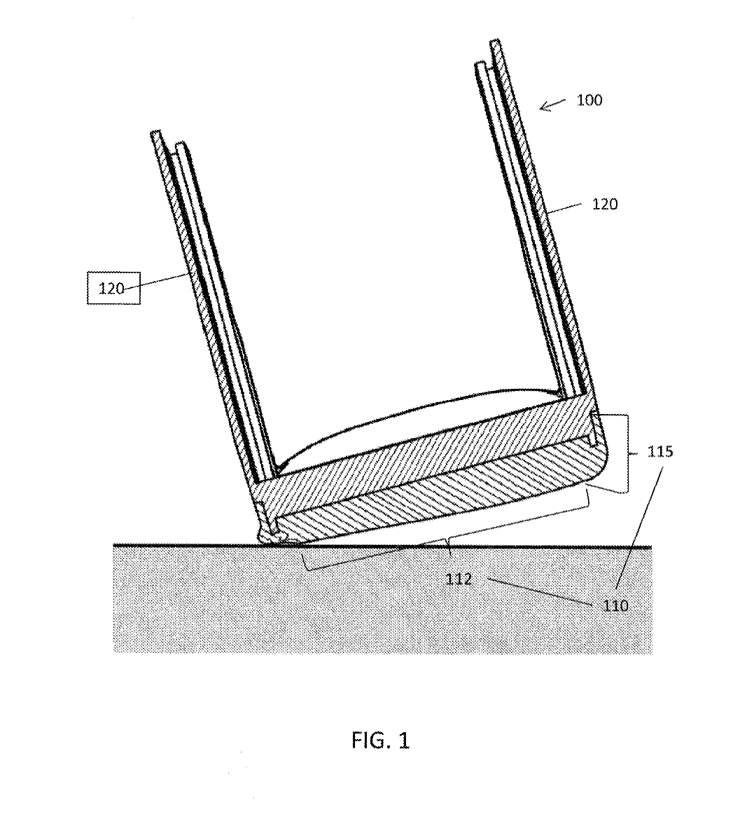

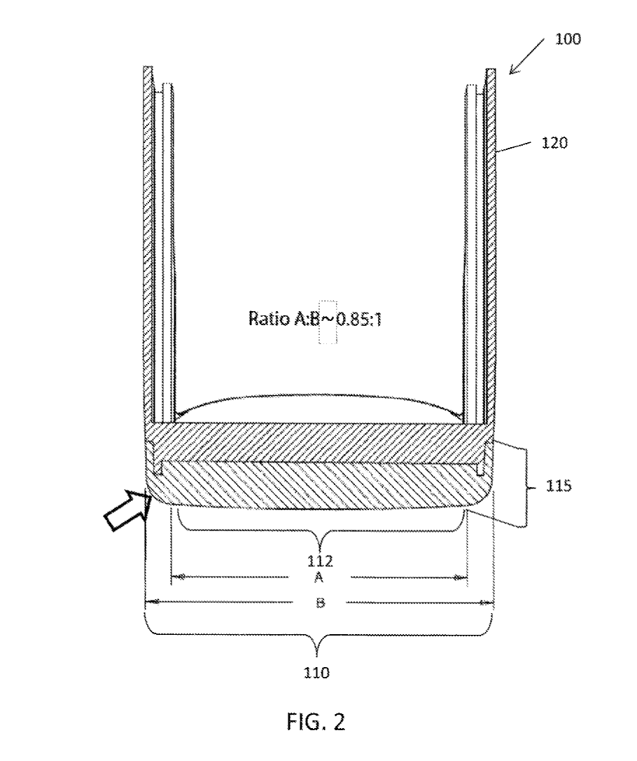

FIGS. 1 and 2, depict an exemplary embodiment of an orthopedic walking boot 100 having a sole 110 with an edge 115 is shown tilted at an angle, such as may occur when a user wishes to pivot to turn, rather than walk in a straight line. The curvature of the sole 110 at either side includes an arcuate edge 115; however, the radius of curvature of the arcuate edge 115 may be so small that the contact surface of the sole with the ground at the arcuate edge 115 is too small to afford the user stability in the effort to turn or may even hinder the turn due to effectively balancing on an edge, e.g., like a skate blade. As shown in FIG. 2, a ratio of the dimension of a substantially flat portion 112 of the sole 110 having a lateral dimension A to a total lateral dimension B, including the two arcuate edges 115, may be on the order of 0.85 or greater, meaning that the sole is mostly or substantially flat over 85% of the surface of the sole that may make contact with a walking surface. A limited portion at the arcuate edges 115 having curvature upward toward an upper portion 120 of the boot 100 at the sides makes actual contact with the ground. In this configuration, the shape of the sole tends to resist the effort to pivot into the turn, adding stress and discomfort to the user's leg, which may adversely affect recuperation.

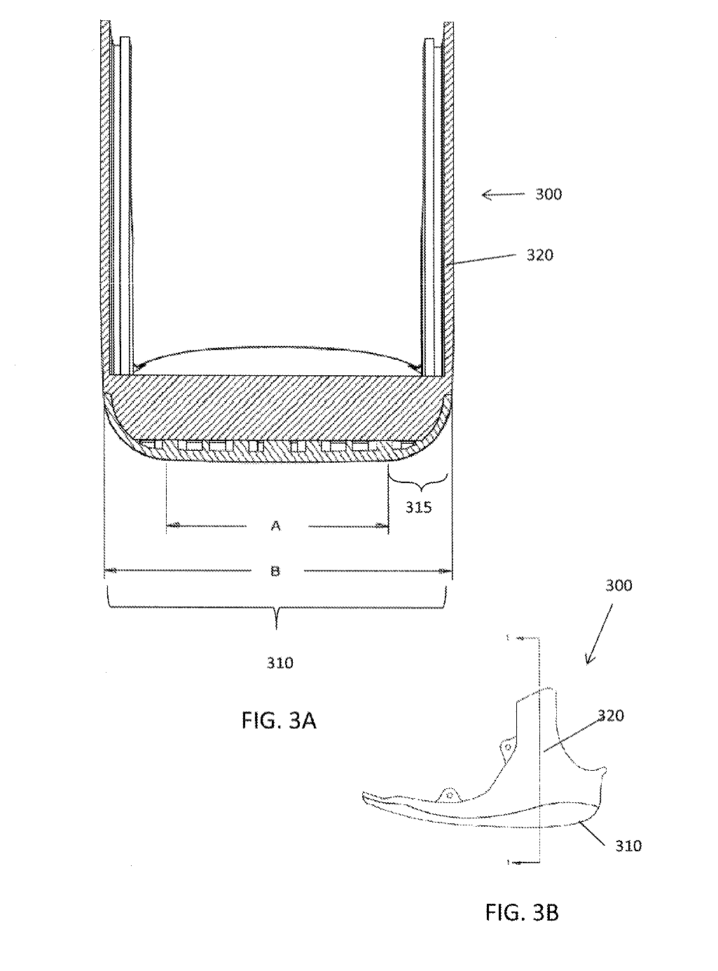

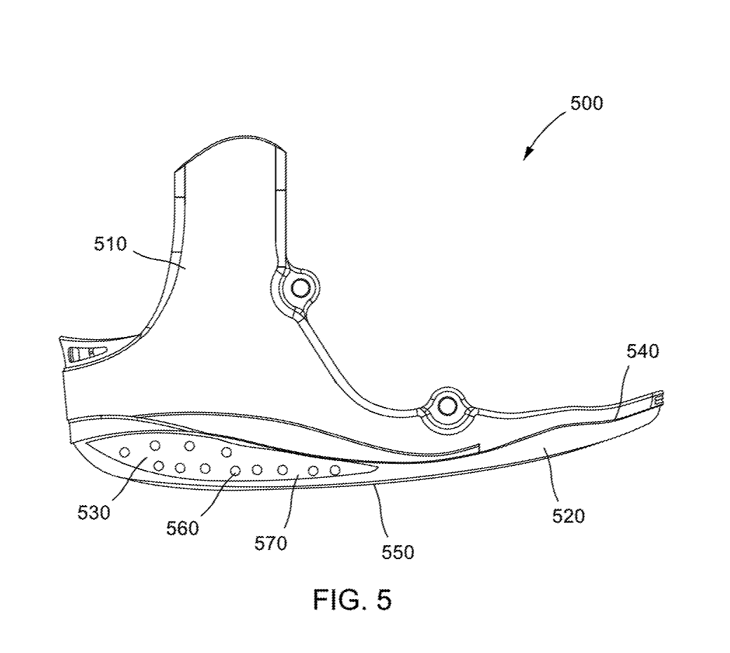

FIG. 3A is a cross-section of an exemplary embodiment of an orthopedic walking boot 300 for a lower portion of a user's leg, i.e., from the knee down. As shown in FIG. 3A, a full length outer sole 310 may be rounded at the sides with an arcuate edge 315 where it comes in contact with the walking surface so that the surface of the boot in contact with the ground may transition more smoothly at the edges than may be encountered in an orthopedic walking boot 100. The rounded or arcuate edge 315 disclosed herein allows for the patient to lean more from side to side in the walking boot to maneuver more easily. With this feature, pivotal rotation of the booted foot is made easier to execute when the user wishes to turn. All portions of the outer sole 310, including the arcuate edge 315, may be fully capable of bearing the weight of the user. The outer soles are either a die cut or injection molded piece of rubber, polyurethane, thermoplastic, rubber, foam, gel/silicone or like material that is attached permanently to a frame of the walking boot. The materials may be used in combination to provide both structural support and stability as well as shock absorption properties, primarily at the heel. Additionally, material mixtures may be utilized for the outer sole to reduce weight, increase strength, enhance traction/grip properties, balance the weight of the walking boot, and/or provide a more desirable thickness that does not suffer from some of the complications discussed above. Material compositions and/or configurations for the walking boot will be discussed in greater detail with respect to FIGS. 5-20.

These outer soles may be sloped in the front-to-back direction, as shown in FIG.

3B, for a front-to-back, or longitudinal, rocking action. These outer soles may be rounded in the front-to-back direction, but not arcuate or rounded in the lateral aspect of the outer sole 310, as shown in FIG. 3A. Because the boot 100 of FIGS. 1 and 2 may usually have a hard angled edge on the lateral sides of the outer sole, the patient, in an effort to assume a natural gait, will actually ride along a portion of the edge, like an ice skate blade, during part of the gait cycle. The problem arises when the patient is on uneven ground, wet surfaces or otherwise unstable surfaces. Because the patient is actually balancing on an edge, it may be more likely that edge becomes cause for the walking boot to slip, providing a dangerous situation for an already compromised, injured, sometimes weakened, or aged patient.

Because the foot and ankle are set at a specified angle, which may be variable, but may be a fixed 90 degree angle, the injured patient may normally adjust his/her gait to not only the frozen angle of the ankle, but to accommodate simultaneously for an angular relationship of the hip to the knee. This causes gross adjustments to the gait/walking patterns, including when the patient pivots to execute a turn. The curved or rounded edge—the arcuate edge 315—will allow for the patient to intuitively adapt to a more normal 3-dimensional gait pattern by being able to roll or use the edge 315 of the walking boot by leaning the body more side-to-side, as in a healthy walking gait, thus accommodating for the injury as well as the ankle being frozen in a 90 degree angle.

The patient may be more comfortable from the first strides when attempting ambulation in the walking boot 300. The walking boot may be beneficially used in a very wide range of injuries and a very wide spectral profile of patient disabilities, e.g., age, physical fitness and/or disabilities, and injury types. For example, a teenage athlete with a broken leg has a very different gate requirement and pain tolerance than an elderly, overweight, health compromised senior citizen who may also suffer other multiple chronic conditions (e.g., arthritis, hip and knee joint degradation, etc.) that can have an additional dramatic effect on gait requirements.

As the injuries progress in healing, the gait pattern may become more aggressive as the pain is eliminated when using the walking boot 300. Because the patient may be more comfortable at all stages of recuperation, he/she may want to adapt a more natural gait, e.g., walking, twisting, turning quickly, etc. Conscious thought is rarely given to the process of walking in our normal lives. However, people will constantly pivot around a chair, twist when exiting a car, and pivot during normal walking activity when maneuvering around or away from objects and corners, i.e., negotiating normal environments such as the household or work, changing walking surfaces, such as from carpet to hard surfaces, etc. A walking boot with a sharp cornered arcuate side edge tends to force a wearer to walk in a straight line along that edge. A walking boot with a more rounded curved side edge allows for less restricted freedom to maneuver more easily. An outer sole with a relatively sharp cornered edge may result in the patient teetering and occasionally slipping or sliding on the edge. The curved surface disclosed herein allows for an easier pivot, roll, turn twist, etc., and improved contact traction on substantially any condition of the walking surface, e.g., snow, ice, rain, oily/slippery surfaces, gravel, rocks, stairs, curbs, all the surfaces we may consistently maneuver on in normal ambulatory activity, to which barely any thought is normally given.

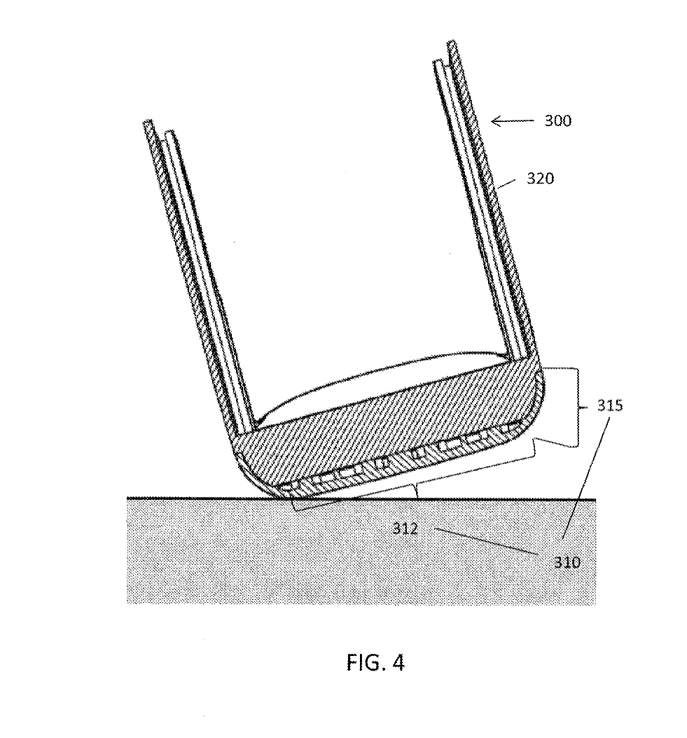

Referring to FIG. 4, the arcuate nature of the walking boot outer sole 310 may have a lateral profile that is continuous across the width of the entire outer sole 310 and terminate without a substantial step-off between the lateral portion 312 and the arcuate edge 315, thereby being congruent with substantially continuous curvature over the entirety of the outer sole lateral surface profile from the heel striking area to the toe. That is, a lateral portion 312 of lateral dimension A of the sole 315 may have a radius of curvature in the lateral plane that is large enough so as to appear that the lateral portion 312 is approximately or substantially flat. The arcuate edges 315 may have a radius of curvature in a smaller range which is, however still larger than for the arcuate edges 115 of the walking boot sole 110.

A total dimension B, includes the lateral portion 312 plus the two arcuate edges 315. At the interface between the approximately or substantially flat lateral portion and each of the two arcuate edges 315 the radius of curvature changes to a smaller value, however the surface of the sole has a transition from one portion to the other, with no substantial discontinuous break in contour between the two parts (i.e., between the substantially flat or slightly curved lateral portion of dimension A and the arcuate edge 315) corresponding to a change in slope of the contour break of no more than 20 degrees. Thus, the step-off between the lateral portion 312 and the arcuate edge 315 is restricted to be equal or less than 20 degrees. For example, the radius of curvature may transition between approximately 10 mm in the region of the arcuate edge 315 to a larger value—up to infinity—in the lateral portion 312 of the outer sole 310 indicated by the dimensions A, provided there is no substantial cusp or discontinuity greater than 20 degrees of the surface smoothness from one portion to the other. More preferably, the radius of curvature in the region of the arcuate edge 315 may be approximately 30 cm. This may vary, for example, according to boot size.

A value of the radius of curvature of infinity in the lateral portion 312 indicates a flat portion of the outer sole 310. The radius of curvature in the lateral portion 312 may be in a first range of values from a minimum specified value up to infinity. The radius of curvature in the arcuate edge 315 may be in a second range of values from, for example, the minimum value specified for the lateral portion 312 down to a smaller specified value. The substantially continuous curvature over the entirety of the outer sole lateral surface profile determines that the lateral contour of the outer sole 310 changes smoothly from lateral portion 312 to arcuate edge 315, i.e., with no sharp edges greater than, for example, a 20 degree step-off.

It may be understood that a tread pattern in the surface of the outer sole 310 may be considered as a perturbation of the surface of the outer sole 310, and is not considered in the definition of the radius of curvature.

Commonly, the ratio may be A:B˜0.85. In the walking boot 300 the ratio A:B may be lower, e.g., on the order of 0.85>A:B≧0. More preferably the ratio A:B may be A:B˜0.63.

Another feature is a scalloping or curved recessing on the inside of the walking boot in the support assembly 320 to accommodate the ankle. Various embodiments of the walking boot 300 disclosed herein may have a curved or recessed inner surface (not shown) of the support assembly 320 which accommodates the natural curvature of the ankle and foot. This provides a pre-relieved area or recess to accommodate the boney prominences of the foot and ankle and also accommodates swelling patterns that are predictably present with injuries to the area.

In yet another embodiment of the orthopedic walking boot, the support assembly 320, may be flared outwardly (not shown) to conform to a shape of the wearer's calf, which has an increasing cross-section of the leg with distance from the ankle.

In addition to the curvature and angle considerations for enhancing a user's experience with the walking boot discussed above, different materials may be incorporated into the outer sole of the walking boot to further enhance the user's comfort when using the walking boot. For instance, different combinations of materials may provide the benefits of better structural integrity, while also fine tuning the design to more closely enable the user of the walking boot to achieve a more comfortable gait, while immobilized.

The outer sole may include a plurality of materials such as, for example, a primary material for structural strength, and one or more secondary materials configured to provide a greater degree of shock absorption to reduce impact stress on the user's foot, particularly from the heal to the mid-foot. The primary and secondary materials may be structurally distinct and separate over the extent of the outer sole to provide different impact characteristics according to location, or alternatively a mixture in various proportions of the primary and secondary materials may provide differing degrees of shock absorption at different locations of the sole of the foot. The mixture may be achieved by controlled additive mixing of secondary materials.

As will be described in the foregoing paragraphs, a walking boot having an outer sole comprising a plurality of materials provides the capability of absorbing the shock involved during heel strike while having a rigidity and durability to also provide the rocker sole shape with the rigidity and durability needed to traverse a variety of surfaces on a stable balanced platform. Using two distinctly different materials can provide a light weight, sturdy low deforming pre-determined shaped surface to walk on with enough durability to support patient of most shapes and sizes. The configurations described below provide added comfort in heel strike and midfoot shuffling.

The use of materials allows the outer sole to provide a light weight, low profile, durable cushioned sole that provides comfort to the user. Utilizing materials that absorb shock reduce the pain associated with the force generated by the heel strike, because the material is energy absorbent. Utilizing materials for rigidity and durability enable a user of the walking boot to comfortably traverse a variety of surfaces on a stable, balanced platform. As will be described in the foregoing, several different combinations of materials of varying strengths and thickness may be used to achieve a normal gait pattern while minimizing pain and discomfort.

FIG. 5 is a side view of an exemplary embodiment of an orthopedic walking boot 500 comprising an outer sole comprising a plurality of materials. The orthopedic walking boot 500 comprises a support structure 510 similar to the support structure 320, a base 540, an outer sole first material 550, and voids 560. The outer sole 550 comprises a toe region 520 and a heel region 530. The heal region 530 comprises a second material 570. As shown, the base 540 is arranged with the outer sole 550.

In this exemplary embodiment, the lower or external portion of the outer sole 550 may be comprised of a first material and enclose a second material 570 in the heel region 530. The first and second materials may comprise Thermoplastic Elastomer (TPE) of varying hardness. One example of a TPE material may be PolyOne TPE AC425.

The first material may have a hardness of between 45 and 90 Shore A. This first material provides structural strength and abrasion resistance along the entire external surface of the walking boot. The first material is designed to be of a uniform thickness between 3 mm and 6 mm. In some aspects of the outer sole, the voids 560 may provide visibility to the second material. However, the voids 560 are optional to the configuration illustrated in FIG. 5. Unlike the first material, the second material 570 is formulated to have shock absorption qualities. The second material 570 may have a hardness between 10 and 75 Shore A. The thickness of the second material 570 may vary due to molding constraints. Additionally, the thickness of the second material 570 may vary due to molding constraints as well as built in voids 560 to fine tune the shock absorption properties of the material. For instance, a thicker second material 570 may provide greater shock absorbing properties.

The second material 570 may be made as a separate insert and placed into the base before molding the first material of the outer sole 550 over it. Alternatively, the second material 570 and the first material of the outer sole 550 may be co-molded to the base 540, where they are both injected onto the base consecutively (e.g., the second material 570 followed by the first material).

Using TPE as the first and second materials offers flexibility in terms of performance per application. The materials can be injected onto each other or otherwise bonded mechanically or chemically. The advantage of injecting the materials directly onto a walking boot base structure, such as the base 540, simplifies the manufacturing process by removing the bonding step that may require additional manufacturing time. Additionally, the mechanical or chemical bond can be more reliable and repeatable in a manufacturing setting.

In some aspects of the walking boot, texture may be added to the surface of either the base 540, or primary and/or secondary materials to promote adhesion. The formulations of the first and second materials can be adjusted to change the properties to coincide with the intended function of the walking boot. For instance a greater proportion of softer material may be used for greater shock absorption while a greater proportion of harder materials may be used for added structural support. Texture and/or geometric features may be added to the bottom surface of the outer sole to promote traction to different surfaces. The addition of texture and/or geometric features may limit the stickiness, slipperiness, or noisiness commonly associated with materials like TPE.

The texture and/or geometric features help to limit direct contact of the material to a surface that a user may be walking on with the orthopedic walking boot.

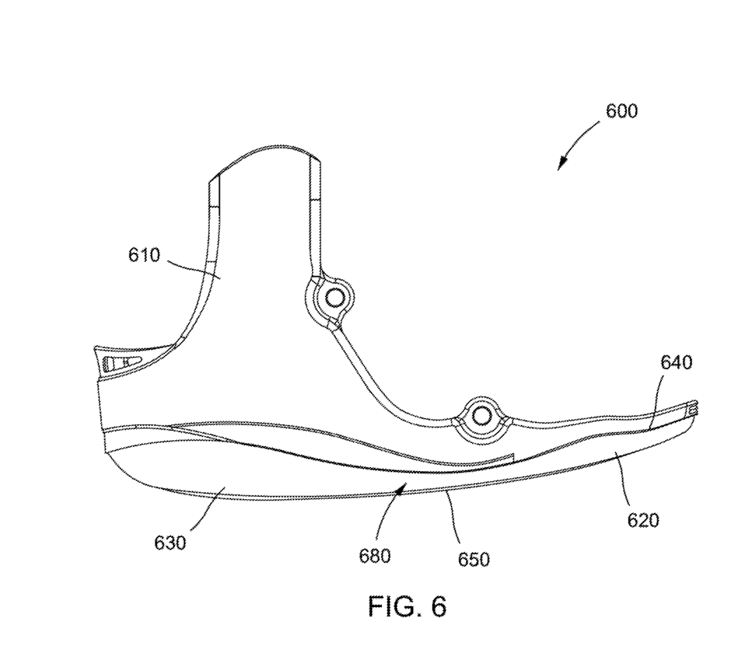

FIG. 6 is a side view of an exemplary embodiment of an orthopedic walking boot 600 comprising an outer sole comprising a plurality of materials. The orthopedic walking boot 600 comprises a support structure 610 similar to the support structure 320, a base 640, and an outer sole 650. The outer sole 650 comprises a toe region 620, a heel region 630, and a transition region 680.

The outer sole 650 may include a first material and a second material. For instance, to maintain rigidity, the toe region 620 may include a larger volume of harder material, while the heal region 630 may include a larger volume of a softer material for shock absorption properties.

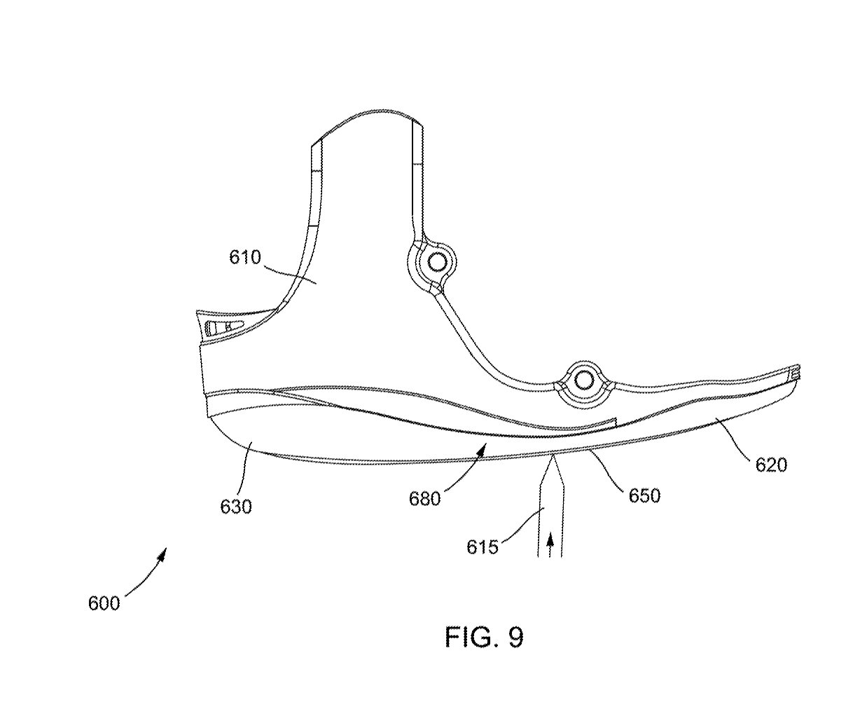

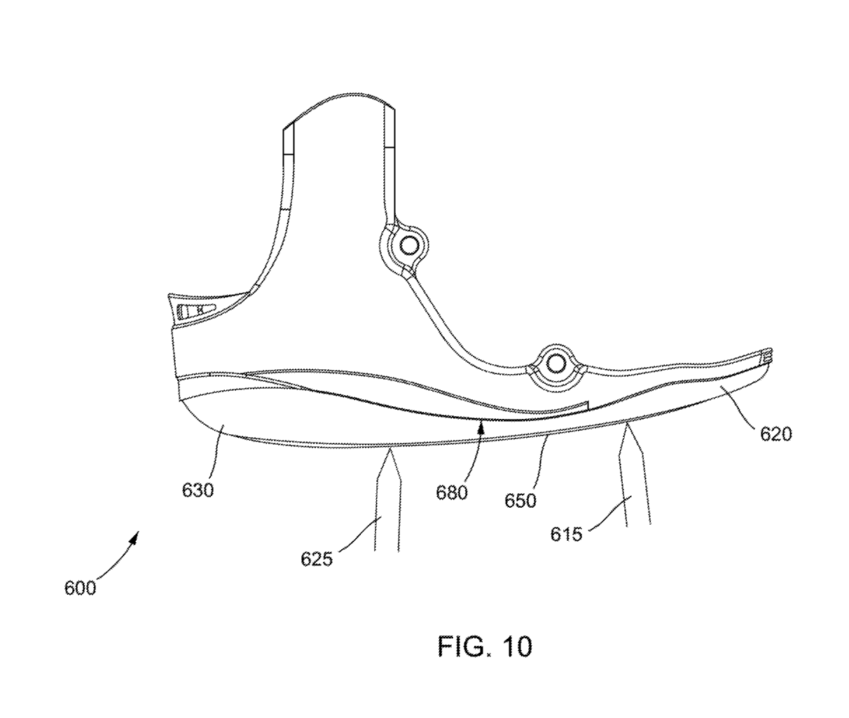

The first and second materials may be Thermoplastic Polyurethane (TPU). An example of a TPU that may be used for the outer sole is BASF Elastollan. The TPU may be injected directly onto the base 640 of the orthopedic walking boot 600. In this exemplary figure, the composition of the TPU changes as it fills from the toe region 620 to the transition region 680, around a mid-foot region of the outer sole, to heel area 630. The composition is controlled by regulating the ratio of harder TPU pellets in an injection machine (see FIG. 9) or by injecting the first and second materials through different injectors (see FIG. 10). By using such injection techniques, the TPU composition provides different properties in the toe region 620 as it does in the mid foot to heel region 630. For instance, the outer sole 650 may comprise a higher concentration of the first material in the toe region 620. At the transition region 680, the composition may transition from the higher concentration of the first material to a higher concentration of the second material in the heel region 630. In this instance, the first material may be harder than the second material so that the first material provides greater structural support in the toe region 620 and greater shock absorption in the heel region 630. Such a composition, in combination with the rocker design discussed above provides a more natural gate for a user having an immobilized foot and ankle.

The advantage of a higher concentration of the softer material in the mid foot to heel region 630 yields better shock absorption during heel strike. Better shock absorption diminishes pain associated with walking with an injury and increases comfort. The harder material provides structural strength as well as add better abrasion resistance to increase product life and traction. The combination of the first and second materials provides greater comfort along with a more natural gait for an injured user.

In this exemplary embodiment, the thickness of the second material at the heel region 630 may be greater than the thickness of the first material in the toe region 620. As a result, greater shock absorption properties are realized at the heal region 630 rather than the toe region 620.

As with FIG. 5, adding texture or surface features/geometry to the bottom of the outer sole 650 will promote adhesion to the ground. The overall thickness of the outer sole 650 can be minimized to a thickness of about 3 mm the first and second materials into a single layer. As a result heel height of the orthopedic walking boot may be reduced along with the weight of the orthopedic walking boot.

The first and second materials may be molded onto the base 640 as illustrated in

FIG. 6 or molded separately from the walking boot base and bonded mechanically or chemically to the base.

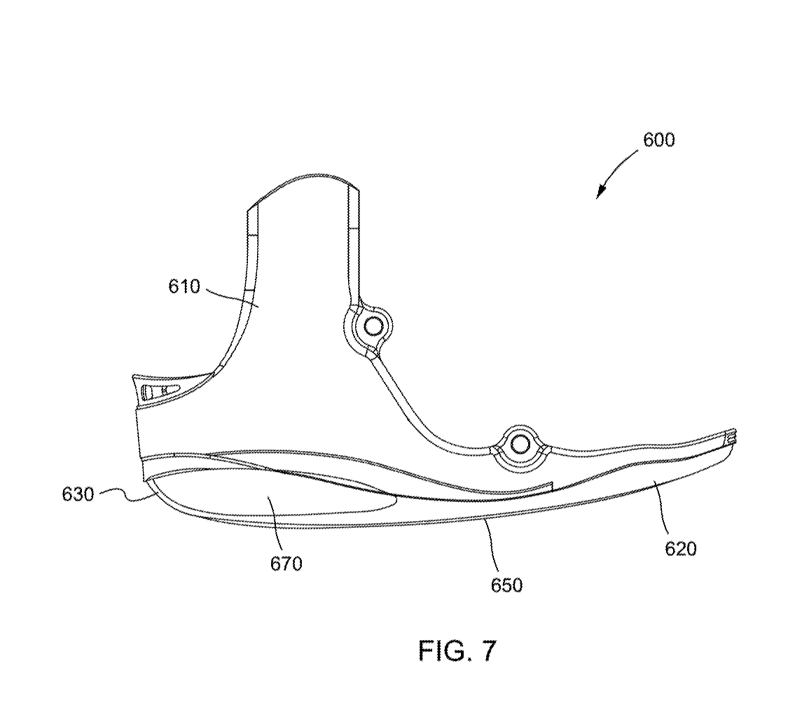

FIG. 7 is a side view of an exemplary embodiment of an orthopedic walking boot 600 comprising the same plurality of materials described with respect to FIG. 6. As shown, the outer sole 650 also includes a region 670 of the second material to further enhance shock absorption. The outer sole 650 may further include a composition of the first and second materials that varies throughout the toe region 620 and the heal region 630 to enhance shock absorption and structural strength.

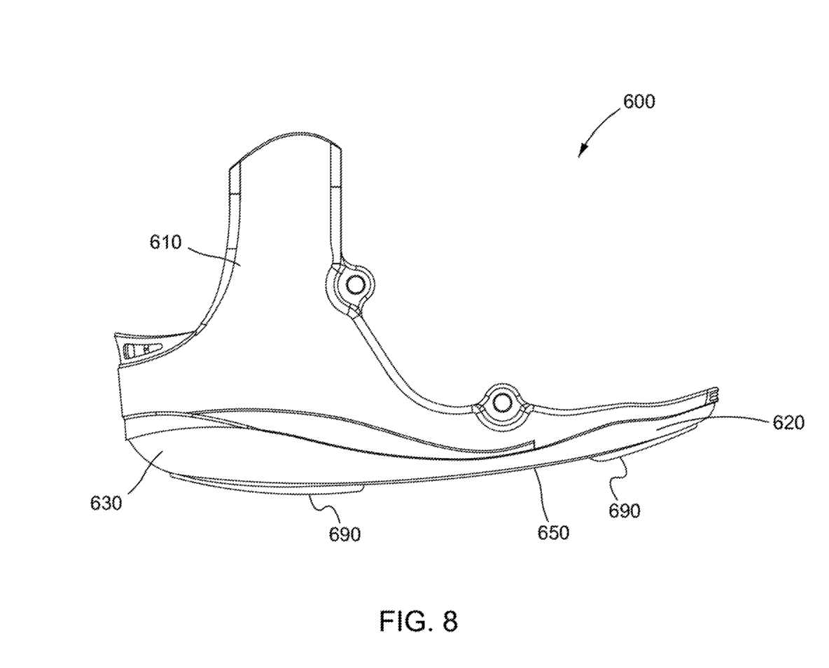

FIG. 8 is a side view of an exemplary embodiment of an orthopedic walking boot 600 comprising the same plurality of materials described with respect to FIG. 6. As shown, the regions 690 comprise at least a portion of the toe and heel regions 620 and 630 of the outer sole include the first material, while the rest of the outer sole 650 includes a mixture of the first and second materials. The mixture may surround the regions 690, while leaving at least a portion of the first material exposed. Again, texture and/or geometric features may be added to base 650 and/or the first and second materials to promote a mechanical bond.

FIG. 9 illustrates an exemplary embodiment of regulating the ratio of harder material pellets in an injection machine to form the outer sole 650 of the orthopedic walking boot. As shown, a nozzle 615 may inject a composition of material such as TPU that varies in hardness by adjusting the amount of harder TPU pellets (the first material) as the nozzle 615 injects material from the heel region 620 through the transition region 680 to the heel region 630. As discussed above, the composition may include an increasing volume of pellets of softer material as the nozzle injects material toward the heel region 630.

FIG. 10 illustrates an exemplary embodiment of injecting the first and second materials through different injector nozzles. As shown, the injector nozzles 615 and 625 dispense compositions of the first and second materials. However, the nozzle 615 may inject a composition have a larger volume of the first material around the toe region 620 to the gradient region 680. The nozzle 625 may inject a composition having a larger volume of the second material around the heel region 630 to the gradient region 680.

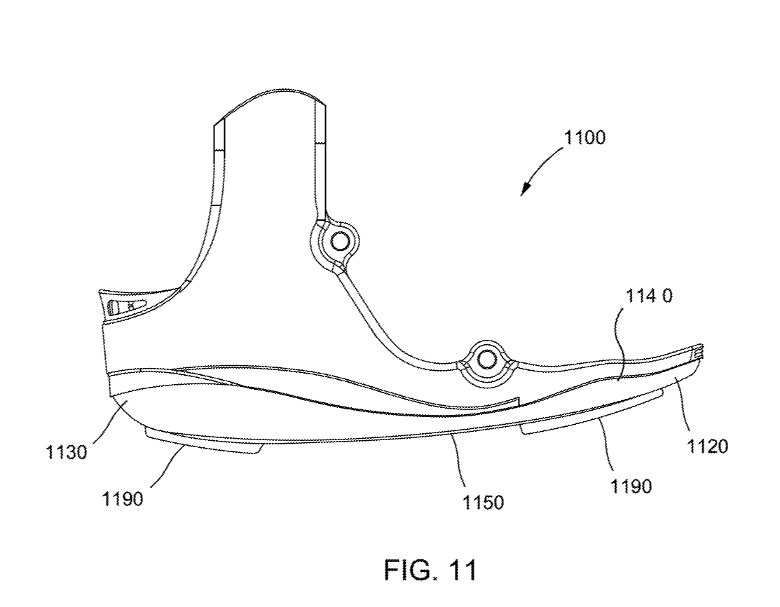

FIG. 11 is a side view of an exemplary embodiment of an orthopedic walking boot 1100 having an outer sole comprising a plurality of materials. As shown, the orthopedic walking boot 1100 includes an outer sole 1150 arranged with a base 1140. The outer sole 1150 includes a toe region 1120 and a heel region 1130. At least a portion of the toe region 1120 and the heel region 1130 includes a first material 1190. An exemplary rubber material may be Vibram Mont. The heel region 1130 of the outer sole 1150 may also include a second material such as Ethylene-vinyl acetate (EVA) or foam. EVA produces materials which are rubber-like in softness and flexibility. An exemplary EVA material is Dupont Elvax EVA. Rubber materials can be classified as natural rubber or synthetic rubber. One advantage of using rubber material is that it has good abrasion resistance properties. Further, rubber material has a high coefficient of friction so it can be used for traction and grip. Rubber material is also flexible so it can conform well to adjoining materials, especially when the adjoining materials can deform. EVA material can be used as a shock absorber, for example EVA with a range of durometers 10-75 Shore A.

Closed cell foam/EVA is preferred because it is highly water resistant and avoids moisture. As will be discussed in the foregoing, rubber and EVA.

When rubber material is mechanically attached to an EVA/foam, the rubber material will promote its abrasive resistance and traction to the outsole and the EVA will promote its shock absorption. Rubber is commonly heavier than EVA, so using less material for the rubber and more material of the EVA/foam will reduce total weight of the outsole.

Shock absorption is commonly needed in the heel region 1130 to the midfoot so more volume of the EVA/foam material is used in the heel region 1130 to a midfoot region rather than the toe region 1120. For instance, the EVA thickness on the heel region may be 20 mm and the thickness of the toe region may be 5 mm. An area for abrasion resistance on the outsole may be utilized all around the outsole especially on the heel region 1130 and the toe region 1120, likewise for traction and grip. The rubber material may be mechanically attached around the heel region 1130 and the toe region 1120. For example the first material may have a thickness of 3 mm to avoid increasing the weight of the outer sole, thereby increasing the overall weight of the walking boot.

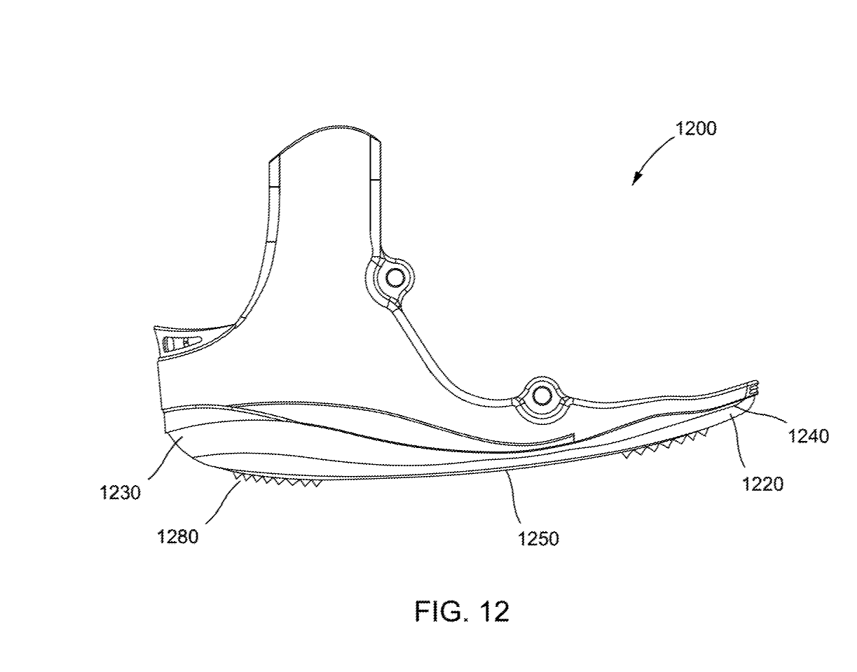

FIG. 12 is a side view of an exemplary embodiment of an orthopedic walking boot 1200 having an outer sole comprising a plurality of materials. The walking boot 1200 includes a base 1240 and an outer, a toe region 1220, a heel region 1230, and surface texturing 1280. The outer sole 1250 comprises a composition of EVA/foams having first and second durometers (hardness). One of the advantages of using EVA/foam for the majority of the outer sole 1250 is that the weight of the outer sole 1250 may be reduced. A reduction in the weight of the outer sole 1250 provides the capability of a more natural gait for a user of the walking boot 1200. EVA/foam material also has a longer lifetime, thereby increasing the lifetime of the walking boot.

A combination of EVA/foam materials with different durometers attached chemically or mechanically may be configured to provide both greater structural strength and shock absorption. For instance more EVA/foam with a higher durometer may be used more in the toe region 1220 while more EVA/foam with a lower durometer may be used in the heel region 1230. The EVA/foam having a higher durometer may be a first material of the outer sole 1250, while the EVA/foam having a lower durometer may be a second material of the outer sole 1250. The first and second materials may be layered. For instance, the second material may be layered above the base 1240, while the first material may be layered over the second material.

In this instance, the first material may comprise the textured surface 1280 to provide better traction and grip for a more natural gait. The textured surface may comprise different shapes and/or patterns such as a plurality of fins located in varying portions of the outer sole 1250 such as at the toe region 1220 and the heel region 1230.

Since the second material comprises EVA having a lower durometer, the second material will provide shock absorption properties. In one aspect of the walking boot, the EVA may be blended with other materials for additional benefits. For instance, to increase its shock absorbency properties the EVA of the second material may be blended with TPV. The second material may be used more, in terms of volume, in the heel region 1230 and midfoot area than the toe region 1220 because shock absorption is more critical during the heel strike phase of a user's gate. This is because the most force is applied against the user's foot, which may induce greater pain on the user's injury. Good shock absorption can reduce the pain experienced by a user of the walking boot during the heel strike phase of the user's gait. In some aspects of the walking boot, the first material may fully enclose the second material, to protect the surface of the outer sole from experience abrasions to the less durable second material.

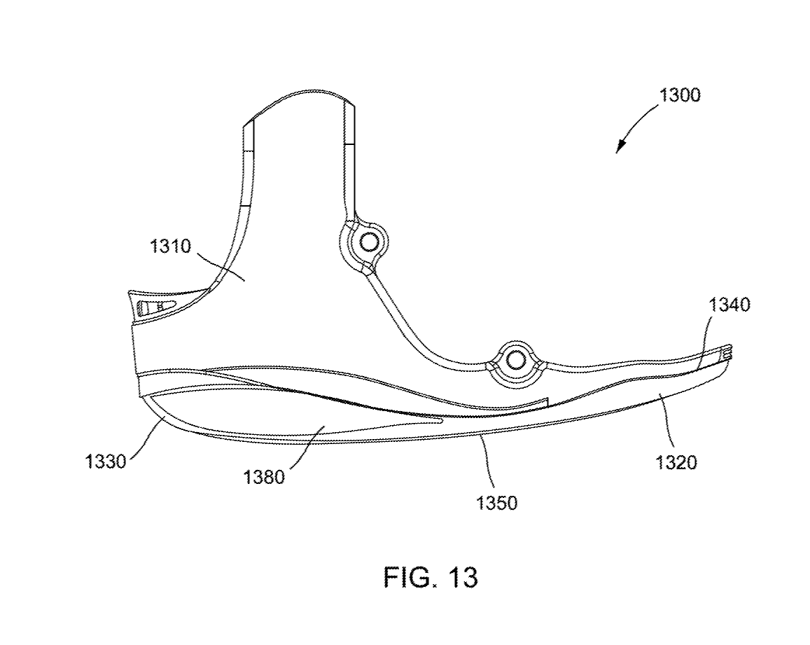

FIG. 13 is a side view of an exemplary embodiment of an orthopedic walking boot 1300 having a plurality of materials in an outer sole. The walking boot 1300 includes a support structure 1310, a base 1340, and an outer sole 1350. The outer sole 1350 may include a toe region 1320, a heel region 1330, and a second material region 1380.

In this exemplary embodiment of the walking boot 1300, the outer sole 1350 may comprise first and second different materials having different durometers. For instance, the first material may comprise TPE. As discussed above, TPE may be used for structural strength and abrasion resistance along the entire surface of the outer sole 1350.

The first material may have a hardness of 45-90 Shore A and may have a greater concentration in the toe region 1320 of the outer sole 1350.

The second material may be TPU, which is formulated to have shock absorption qualities. The second material may have a hardness of 10-75 Shore A and have a greater concentration in the heel region 1330. For instance, the second material may have a high concentration of material in the second material region 1380, as illustrated in FIG. 13.

The first and second materials may be bonded mechanically. Additionally, texture and/or surface features can be used to enhance adhesion and bonding.

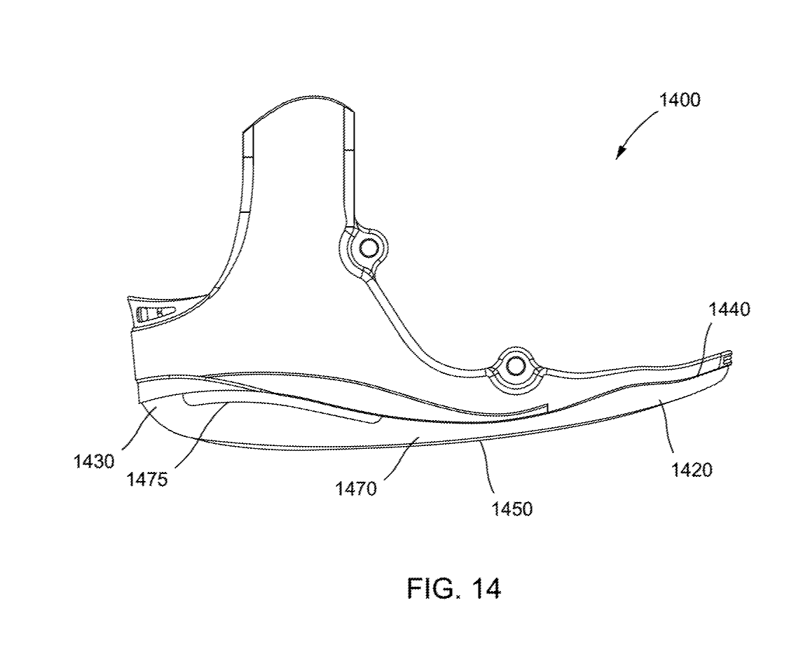

FIG. 14 is a side view of an exemplary embodiment of an orthopedic walking boot 1400 having an outer sole comprised of a plurality of materials. The orthopedic walking boot 1400 includes aa base 1440 and an outer sole 1450 attached to the base. The outer sole 1450 includes a toe region 1420, a heel region 1430, a first material region 1470, and a second material region 1475.

As shown, the first material region 1470 surrounds the second material region 1475. In this exemplary configuration, the first material may comprise TPU and the second material may comprise a gel/silicone. An example of a gel/silicone that may be used in the outer sole 1450 may be RK Rubber Silicone. As discussed above, TPU provides good structural strength for the outer sole 1450, in part because it has a good abrasive resistance. On the other hand, gel and/or silicone provide good shock absorption properties. However gel and silicone are each heavier than TPU. In order to maintain a lightweight outer sole 1450, a lower volume of gel or silicone.

As shown in FIG. 14, the second material region 1475 may comprise the gel/silicone, while the first material region 1470 may comprise the TPU. As shown, a greater volume of TPU versus silicone/gel forms the outer sole 1450. For instance, the gel/silicone material of the second material region 1475 may have a thickness of 5 to 8 mm. This thickness provides good shock absorption properties, making large volumes of gel/silicone unnecessary.

As shown, the gel or silicone material is primarily positioned in the heel region 1430 and midfoot area. The gel or silicone material may be a pod shape, which provides better shock absorption on the heel point. The first material (TPU) may be located anywhere on the outer sole 1450. Since it is fairly light, the TPU material may also fully enclose the gel/silicone material, as illustrated in FIG. 14.

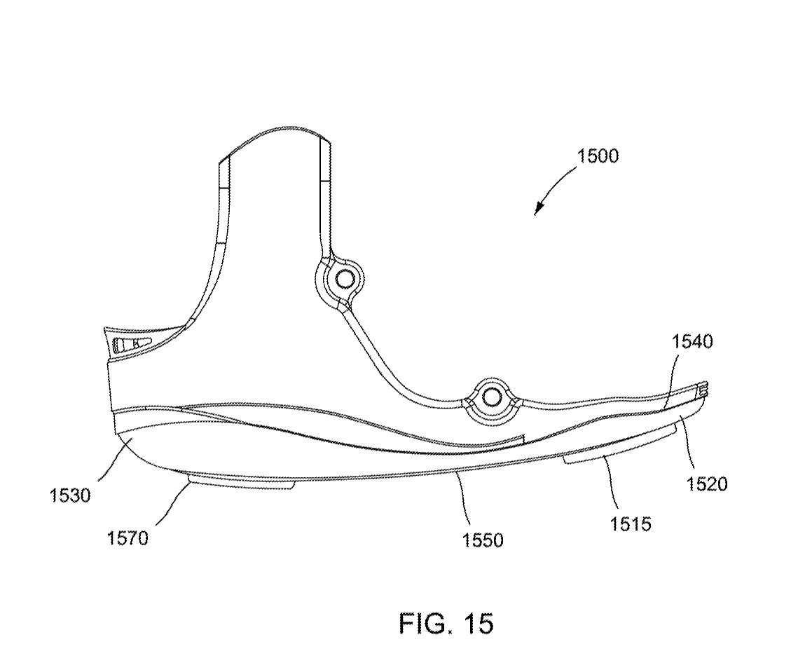

FIG. 15 is a side view of an exemplary embodiment of an orthopedic walking boot 1500 having an outer sole comprised of a plurality of materials. As shown, the walking boot 1500 includes a base 1540 arranged with an outer sole 1550. The outer sole 1550 comprises a toe region 1520, a heel region 1530, and second material regions 1570-1575.

As shown, the outer sole 1550 may comprise first and second materials. The first material may be formed around the second material, while both materials are externally exposed. For instance, the first material may surround but not cover the second material.

In this exemplary configuration, the first material may be a gel or silicone. The second material may be a Styrene-butadiene rubber (SBR). An example of such a rubber may be Robinson Rubber SBR.

In this example a gel or silicone having a high durometer may be used for structural strength purposes. Moreover gel and silicone have good traction and grip properties. Thus using gel or silicone for the first material is beneficial to traction and grip because they have high coefficient of friction. Additionally, incorporating texture and/or adding geometric features on the external surface of the outer sole 1550 adds structural strength and abrasive resistance, while also limiting stickiness.

SBR of a lower durometer has good shock absorption properties. In this exemplary walking boot, the gel or silicone material may be heavier than the SBR, so a lower volume of the gel or silicon compared to the SBR may be used to form the outer sole 1550. For example, the gel or silicone may be used in high wear areas such as the second material regions 1575 and 1570 within the toe region 1520 and heel region 1530. In some aspects of the walking boot, the majority of the material used to form the outer sole may be the second material.

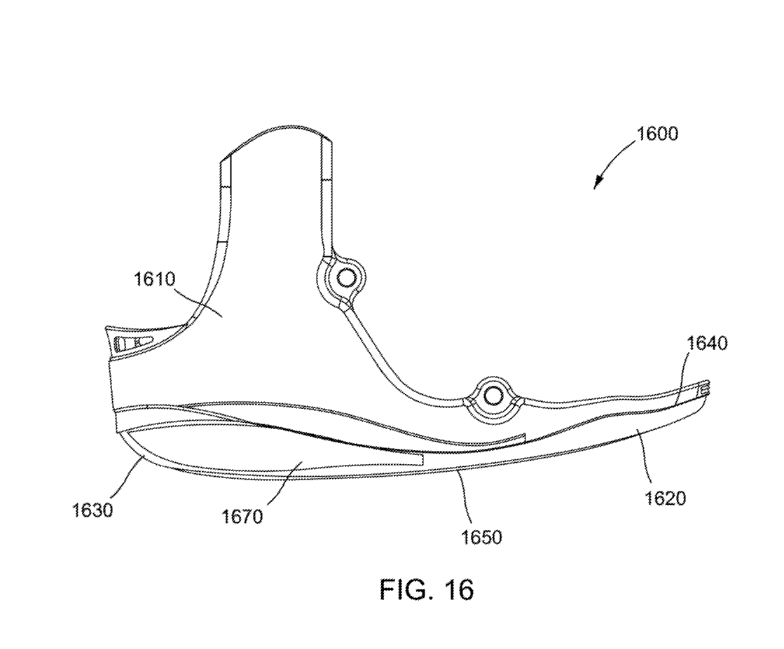

FIG. 16 is a side view of an exemplary embodiment of an orthopedic walking boot 1600 having an outer sole comprised of a plurality of materials. As shown, the walking boot 1600 includes a support structure 1610, a base 1640 arranged with an outer sole 1650. The outer sole 1650 comprises a toe region 1620, a heel region 1630, and a second material region 1670.

As shown, a first material of the outer sole 1650 may surround the second material region 1670. The second material region may comprise the second material. In this exemplary configuration, the first material may be Thermoplastic Vulcanizate(TPV). An example of a TPV material that may be used to form the outer sole 1650 may be

ExxonMobil Santoprene. The TPV material provides structural strength and abrasion resistance along the entire surface of the outer sole 1650.

TPV shares certain properties with rubbers and thermoplastics. TPV provides a good flex life as well as chemical resistance, which provide for a good outer tread surface that protects any internal materials. The TPV of the first material may have a hardness of 45-90 Shore A.

The second material formed in the second material region 1670 may be TPU formulated with a lower hardness to have good shock absorption qualities. The first and second materials may be mechanically bonded. Additionally, texture and surface features and/or geometries can be added to the base 1640 and/or the first or second materials to improve adhesion and bonding. One of ordinary skill in the art will appreciate that alternative materials, such as those discussed above may be used as a shock absorber without departing from the scope of the disclosure. For instance EVA foam, gel, silicone, and rubber such as SBR are a few examples of shock absorbing materials that may be used.

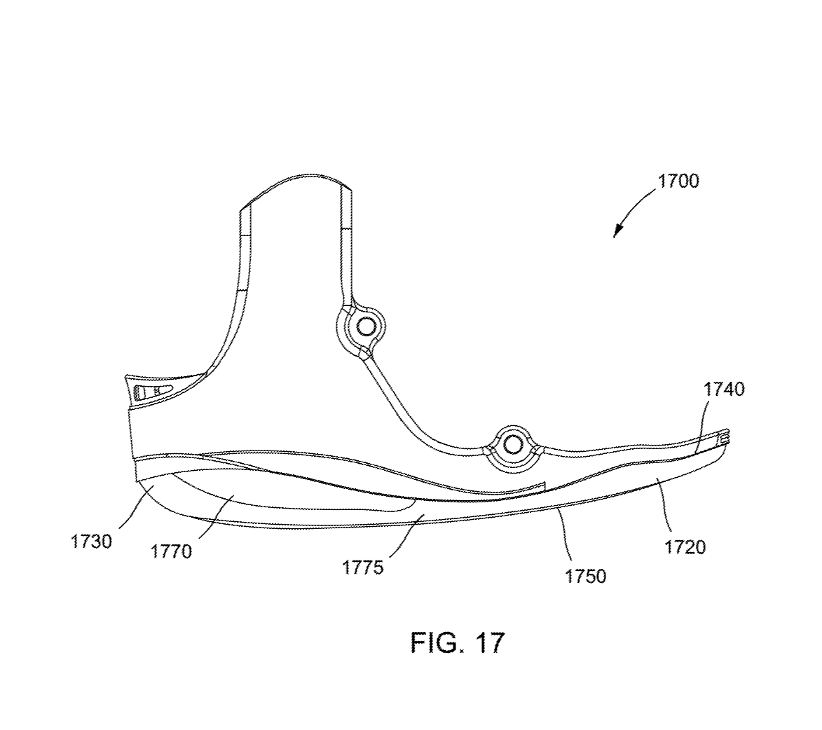

FIG. 17 is a side view of an exemplary embodiment of an orthopedic walking boot 1700 having an outer sole comprised of a plurality of materials. As shown, the walking boot 1700 includes a base 1740 and an outer sole 1750 arranged with the base 1740. The outer sole 1750 includes a toe region 1720, a heel region 1730, a first material region 1775 and a second material region 1770. As shown, the first material region 1775 of the outer sole 1750 may surround the second material region 1770. In this exemplary configuration, the first material may include rubber and the second material may include TPV. As discussed above, rubber material provides good abrasion resistance properties. TPV materials of a lower durometer provide good shock absorption properties.

TPV provides good shock absorption properties because it has a good cycle or flex life. As a result, TPV is less likely to change shape or permanently deform from its original shape (e.g., have a compression set). To lower the weight of rubber/TPV composition, the volume of the second material (the TPV material) may be more than the volume of the first material (the rubber material) in the heel region 1730 to midfoot area. For example, thickness of second material may be between 15 and 20 mm. The thickness of the first material (the rubber material) formed in the heel area may be between 3 and 5 mm.

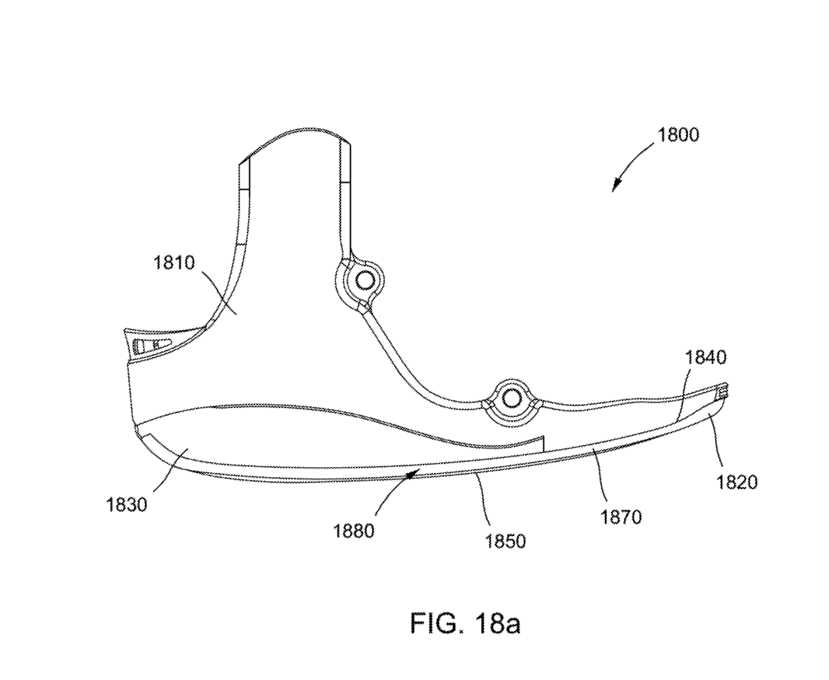

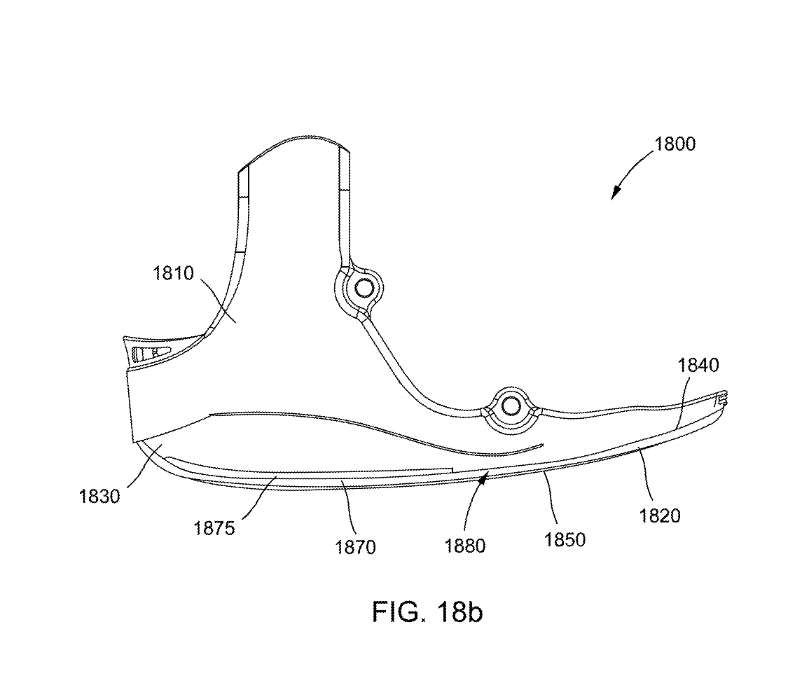

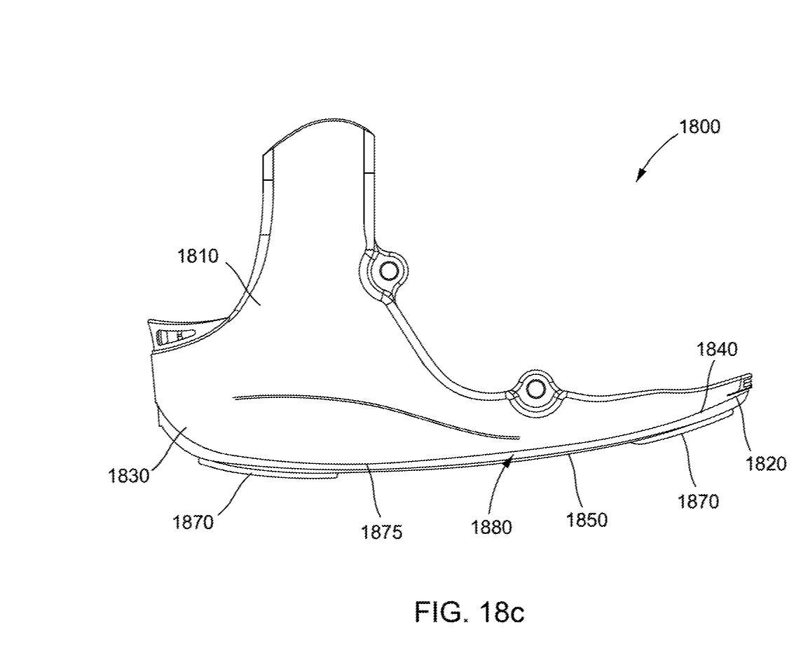

FIGS. 18a-c are side views of an exemplary embodiment of an orthopedic walking boot 1800 having an outer sole comprised of a composition of rubber materials. The walking boot 1800 includes a support structure 1810, a base 1840, and an outer sole 1850 arranged with the base 1840. The outer sole 1850 includes a toe region 1820, a heel region 1830, a first material region 1870, second material region 1875, and a transition region 1880.

As shown in FIG. 18a, a first material may be directly molded onto the bottom of the orthopedic walking boot 1800. In some aspects of the walking boot, the first and second materials may be different rubbers. An example of a rubber material that may be used to form the outer sole 1850 may be Vibram XS Trek. Rubber is capable of providing structural strength and abrasion resistance.

Rubber materials may be classified as natural rubber or synthetic rubber. As shown, the composition of raw rubber may change as it fills from the toe region 1820, through the transition region 1880, located near the middle of base of the walking boot, to the heel region 1830. The transition region 1880 may be where the composition of the rubber mixture changes between having a higher ratio of harder material to having a higher ratio of softer material and vice versa. The composition is controlled by controlling the ratio of harder and softer rubber in the molding process. As a result, the outer sole 1850 comprises a varying mixture of materials and different properties in the toe region 1820 as it does in the heel region 1830.

As discussed above, the higher concentration of the softer material in the heel region 1830 yields better shock absorption during a heel strike from a user using the walking boot. In some aspects of the walking boot, the thickness of the material at the heel region 1830 may be greater than the thickness at the toe region 1820, in order to provide shock absorption properties at the heel region 1830 rather than the toe region 1820.

Natural rubber such as Polyisoprene is relatively heavy. Minimizing the total thickness and density such a material will keep the walking boot 1800 more lightweight. By mixing the two durometers of the rubber material into one single layer a thinner overall tread on the walking boot 1800 may be realized. This minimizes weight and cost without loss of structural strength, abrasion resistance and shock absorption properties. Additionally, as shown in FIGS. 18b and 18c, second material 1875 may be added to promote shock absorption or structural strength and abrasion resistance relatively.

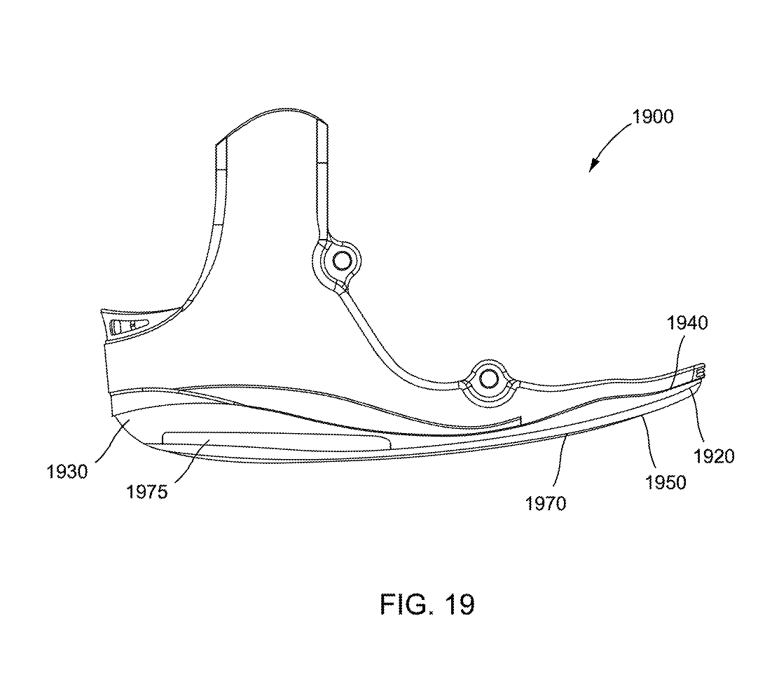

FIG. 19 is a side view of an exemplary embodiment of an orthopedic walking boot 1900 having an outer sole comprised of a plurality of materials. The walking boot 1900 includes an outer sole 1950 arranged with a base 1940. The outer sole includes a toe region 1920, a heel region 1930, a first material region 1970, and a second material region 1975.

As shown, the first material region 1970 is a thin layer of a first material. Formed over the first material region 1970 is the second material region 1975. In some aspects of the walking boot, the first material may be a gel or silicone such as RK Rubber Silicone. The second material may be a gel or silicone such as Northstar Polymers Gel Elastomer. As discussed above, gel and silicone have higher durometers, making them strong materials to use for structural strength purposes and abrasion resistance. This is because gel and silicone have good traction and grip properties. Moreover, gel or silicone of a lower durometer have good shock absorption properties. However, since gel and silicone are heavier materials, both higher and lower durometer gels or silicones should be formed as layers or sheets to reduce the weight. As shown in FIG. 19, both first and second material regions, which each comprise gel or silicone are formed as thin sheets or layers.

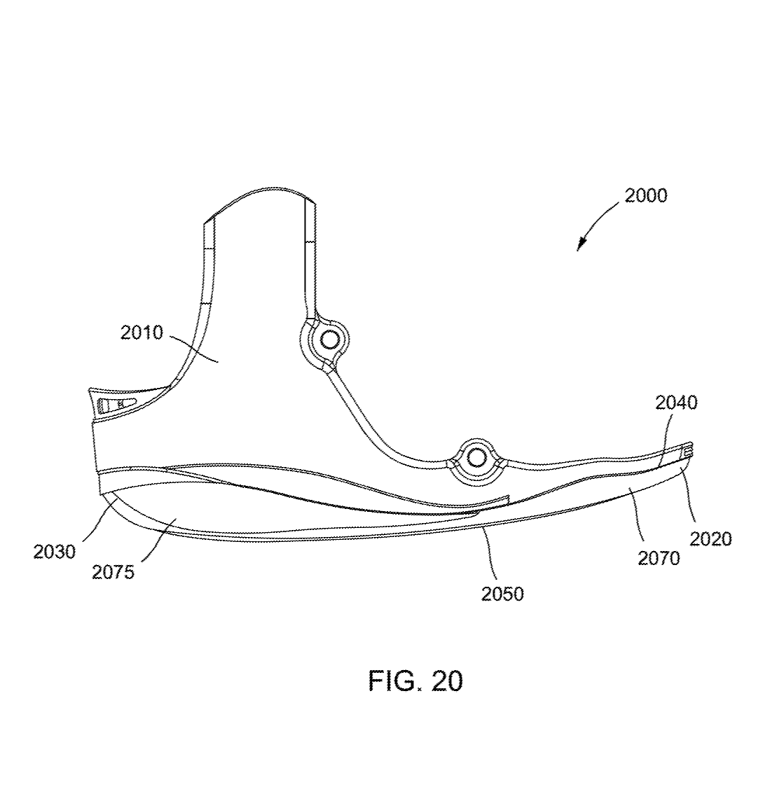

FIG. 20 is a side view of an exemplary embodiment of an orthopedic walking boot 2000 having an outer sole comprised of a plurality of materials. The walking boot 2000 includes a support structure 2010, a base 2040 arranged with the outer sole 2050. The outer sole 2050 includes a toe region 2020, a heel region 2030, a first material region 2070, and a second material region 2075.

As shown, the first material region 2070 comprises a first material and the second material region 2075 comprises a second material. In some aspects of the walking boot, the first material may be TPE and the second material may be EVA. The first material (the TPE) may form an encapsulation around the second material region 2075 and also form the support structure 2010. The first material may have a hardness between 45 and 90 Shore A.

The second material may be used as a shock absorber. The second material may have a hardness between 10 and 75 Shore A. It may be preferable to use closed cell foam because it is water resistant and is sealed from absorbing any debris and fluids. As shown, the second material is located in the second material region 2075, located from the midsole back to the heel region 2030 to promote shock absorption during a heel strike. This increases the user's comfort when immobilized by the walking boot. The TPE also comprises a good tread surface in all traction conditions, unlike the EVA foam, which is specially formulated to work well to dampen forces in the heel region 2030. EVA is lightweight which can reduce the overall weight of the product which allows the walking boot to provide the use with a more comfortable gait. This is because balancing the weight of the walking boot to the shoe on the other uninjured foot as closely as possible promotes a more natural gait.

Any of these materials can be blended for advantageous characteristics and to lessen any disadvantages. On of ordinary skill in the art will appreciate that there may be materials not mentioned in this disclosure that may be substituted for any of the above materials without departing from the scope of the invention. For instance, the first and/or second materials may be selected from a group consisting of Thermoplastic Elastomer (TPE), Thermoplastic Polyurethane (TPU), rubber, Ethylene-Vinyl Acetate (EVA) foam, gel or silicone, Styrene-Butadiene Rubber (SBR), Thermoplastic Vulcanizate (TPV), a rubber mixture, and EVA mixed with TPV.

In still another aspect of the disclosure, shock absorption may be achieved by using any of the lower durometer materials described above in an insole of the walking boot.

It may be readily appreciated that the walking boot as described above may simultaneously solve a number of deficiencies found in the prior art. These deficiencies in the prior art may include, but are not limited to, an inability to accommodate: a user's supination or pronation tendencies, changes in mobility during recovery, the need for postural accommodations including the hip, knee, back and shoulders, and desired freedom of movement on various terrains, such as, but not limited to, stairs and inclines.

The claims are not intended to be limited to the various aspects of this disclosure, but are to be accorded the full scope consistent with the language of the claims. It is noted that specific illustrative embodiments of the invention have been shown in the drawings and described in detail hereinabove. It is to be understood that various changes and modifications may be made without departing from the spirit and scope of the invention. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. §112(f) unless the element is expressly recited using the phrase “means for” or, in the case of a method claim, the element is recited using the phrase “step for.”

What is claimed is:

1. An orthopedic walking boot, comprising:
a base to support a user's foot;
a support assembly extending from the base to support the user's lower leg; and
an outer sole comprising a walking surface having first and second materials, wherein the second material has a greater shock absorbing characteristic less than the first material along at least a portion of the outer sole.

2. The orthopedic walking boot of claim 1, wherein the first material comprises a material selected from a group consisting of Thermoplastic Elastomer (TPE), Thermoplastic Polyurethane (TPU), rubber, Ethylene-Vinyl Acetate (EVA) foam, gel or silicone, Styrene-Butadiene Rubber (SBR), Thermoplastic Vulcanizate (TPV), a rubber mixture, and EVA mixed with TPV.

3. The orthopedic walking boot of claim 1, wherein the second material comprises a material selected from a group consisting of Thermoplastic Elastomer (TPE), Thermoplastic Polyurethane (TPU), rubber, Ethylene-Vinyl Acetate (EVA) foam, gel or silicone, Styrene-Butadiene Rubber (SBR), Thermoplastic Vulcanizate (TPV), a rubber mixture, and EVA mixed with TPV.

4. The orthopedic walking boot of claim 1, wherein the first material has a durometer between 45 and 90 Shore A.

5. The orthopedic walking boot of claim 1, wherein the second material has a durometer between 10 and 75 Shore A.

6. The orthopedic walking boot of claim 1, wherein the first material has a thickness between 3-6 millimeters along said at least a portion of the outer sole 7. The orthopedic walking boot of claim 1, wherein the second material has a thickness of 5-8 millimeters along at least a portion of the outer sole.

7. The orthopedic walking boot of claim 1, wherein the support assembly comprises opposing lateral sides, and wherein the walking surface has a substantially continuously curved portion extending between the lateral sides of the support assembly.

8. The orthopedic walking boot of claim 7, wherein the substantially continuous curved portion comprises opposing arcuate portions each coupled to a corresponding one of the lateral sides and a bottom portion extending between the arcuate portions.

9. The orthopedic walking boot of claim 8, wherein each of the arcuate portions comprises a radius of curvature greater than 10 mm along its entire lateral length.

10. The orthopedic walking boot of claim 8, wherein the bottom portion comprises a radius of curvature that increases along its lateral length from each of the arcuate portions toward a center of the bottom portion until reaching a maximum radius of curvature at the center.

11. The orthopedic boot of claim 1, wherein said at least a portion of the outer sole comprises a heel striking portion.
